(19) United States
(12) Patent Application Publication
(10) Pub. No.: US 2016/0215237 A1
(43) Pub. Date: Jul. 28, 2016

(54) Fabric Softener Composition and The Use Thereof

(71) Applicant: AKZO NOBEL CHEMICALS INTERNATIONAL B.V., Arnhem (NL)

(72) Inventors: Leon Zheng, Shanghai (CN); Amy Xu, Shanghai (CN); Mahnaz Company, Hisings Backa (SE)

(21) Appl. No.: 15/025,927

(22) PCT Filed: Oct. 7, 2014
(86) PCT No.: PCT/EP2014/071371

§ 371 Date: Mar. 30, 2016

(30) Foreign Application Priority Data
Oct. 10, 2013 (CN) PCT/CN2013/084957
Feb. 25, 2014 (EP) 14156503.6

(51) Int. Cl. C11D 3/00; C11D 3/43; C11D 3/30; A61K 8/365; A61K 8/41; A61K 8/46; A61K 8/34; A61K 8/60; C11D 3/34; A61Q 5/12

(52) U.S. Cl. CPC C11D 3/0015; C11D 3/349; C11D 3/43; C11D 3/30; A61Q 5/12; A61K 8/416; A61K 8/46; A61K 8/34; A61K 8/602; A61K 8/365; A61K 2800/592

(57) ABSTRACT

The present invention provides for a fabric softener composition comprising a quaternary ammonium compound, a compound selected from alkoxylated quaternary ammonium compounds, nonionic surfactants, alkylaryl sulfonates, and alkylsulfonate compounds. The composition is in liquid state, and remains clear and stable at or below room temperature. When applied to fabric products, the composition of the present invention exhibits comparable or even better softening performance as compared with those emulsion or paste versions of fabric softener that are commonly used in the art. The present invention also provides for the use of the composition of the present invention to soften fabrics.

### TECHNICAL FIELD

This invention relates to a fabric softener composition, especially to a fabric softener composition comprising quaternary ammonium compounds. It further relates to the use of the composition.

### BACKGROUND

Fabric softeners are also referred to as fabric conditioners. They are compositions containing one or more active substances which are used in the laundering process to provide laundered fabrics with a texture that is smooth, pliable and soft to the touch. Fabric softeners generally contain one or more cationic surfactants in the form of water-insoluble quaternary ammonium compounds in which the ammonium nitrogen atom contains at least two long chain acyclic aliphatic hydrocarbon groups.

As the softeners themselves are often of hydrophobic nature, they are commonly occurring in the form of an emulsion. In the early formulations, soaps were used as emulsifiers. The emulsions are usually opaque, milky fluids. However there are also microemulsions where the droplets of the hydrophobic phase are substantially smaller. The advantage of microemulsions is in the increased ability of the smaller particles to penetrate into the fibers. A mixture of cationic and non-ionic surfactants is often used as an emulsifier. Another approach is using a polymeric network, an emulsion polymer.

U.S. Pat. No. 4,255,484 is directed to a fabric-conditioning article impregnated with a fabric-conditioner composition which includes a fatty glyceride spreading agent to enhance uniform transfer of fabric-conditioning agents from the article to fabrics with which the article is tumbled. The fabric-conditioner composition preferably comprises about 60-80% by weight of a cationic fabric-softener composition and about 5-15% of a selected nonionic surfactant, more particularly, an ethoxylated, stearic acid type surfactant. The fabric-conditioner composition according to U.S. Pat. No. 4,255,484 is likely to be in paste state due to the high amount of the cationic fabric-softener and the absence of effective hydrotropes, and it requires a fatty glyceride spreading agent to enhance uniform transfer of the composition from the article to fabrics.

US 2006264352 is directed to a low solids, high viscosity fabric softener composition that contains minimal or no polymeric additives, exhibits improved softening, stability and viscosity performance properties as desired, wherein the composition comprises a specific rheology modifying fabric softening active and another fabric softening active.

However, there is still need for fabric softener compositions that exhibit good stability. The current fabric softener compositions that are in emulsion, micro-emulsion, and emulsion polymer forms are likely to turn phase separated over time. As a result, the appearance and performance of the compositions will become worse accordingly. There consequently exists a need in the art to provide a fabric softener composition with high stability, specifically the stability in appearance and performance.

### DESCRIPTION OF THE INVENTION

The present invention provides for a fabric softener composition which is in liquid state, and remains clear and stable at or below room temperature. When applied to fabric products, the composition of the present invention exhibits comparable softening performance as compared with those emulsion or paste versions of fabric softener that are commonly used in the art. The present invention also provides for the use of the composition of the present invention to soften fabrics.

It has been found that the problems discussed above were solved by the new fabric softener composition according to the present invention.

Surprisingly, it has been observed that the composition of the present invention was stable over time and temperature changes, remained clear and in liquid state at room temperature, and was recoverable after being subjected to cold conditions. Even after freezing, the composition according to the present invention was found to recover spontaneously into clear state under room temperature, without forming any precipitation. This indicates that the composition of present invention is suitable for use in varies of fabric softening products, especially for those applications requiring quick and efficient cold process preparation which does not includes any heating operation in the preparation process. Specifically, the compositions of the present invention were found capable of withstanding at least three rounds of freeze-thaw test, while still remaining clear state without formation of any precipitation when brought back to room temperature.

Other compounds may be optionally included in the composition of the present invention to provide additional functions. Specifically, an organic solvent, preferably selected from the group consisting of isopropanol, ethanol, benzyl alcohol, glycerin, propylene glycol and mixtures thereof may be added for further stabilizing the liquid composition; acids or bases may be included for maintaining the optimal pH for further stabilization of the composition; in addition, electrolytes, carriers such as water and water-alcohol mixture, and other ingredients such as silicone-based anti-foaming agents, emulsion stabilizers, fragrances, and colors, may be added as well, to achieve the relevant desired properties.

In one aspect of the present invention, a fabric softener composition is provided, which comprises:
20-90 wt %, preferably 30 to 70 wt %, based on the total weight of the fabric softener composition of a quaternary ammonium compound according to formula (I):

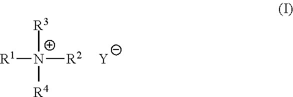

wherein
R¹ and R² are independently a hydrocarbyl group, preferably a linear and saturated or mono-unsaturated hydrocarbyl group, having from 6 to 24, preferably from 8 to 22, more preferably from 12 to 20, most preferably from 14 to 18 carbon atoms;
R³ and R⁴ are independently selected from the group consisting of C₁ to C₃ alkyl, preferably methyl;
Y⁻ is an anion, preferably a halide, such as chloride, or alkylsulphate, such as methyl- or ethylsulphate.
0.01-10 wt %, preferably 0.01-5 wt %, based on the total weight of the fabric softener composition of a compound selected from alkoxylated quaternary ammonium compounds, nonionic surfactants, alkylaryl sulfonates, and alkylsulfonate compounds, wherein the weight ratio of the hydrotope is preferably not greater than 5 wt %; and
0-70 wt % based on the total weight of the fabric softener composition of water.

According to an embodiment of the present invention, the fabric softener composition further comprises 1-60 wt %, preferably 1-50 wt % based on the total weight of the fabric softener composition of an organic solvent, preferably selected from isopropanol, ethanol, benzyl alcohol, glycerin, propylene glycol and mixtures thereof, to further stabilize the resulting liquid composition.

According to an embodiment of the present invention, the fabric softener composition further comprises 0.01-1 wt % based on the total weight of the fabric softener composition of a pH adjusting agent. Preferably, the pH of the composition is at most 7, preferably at most 5, and preferably at least 2.

As used herein, the term alkoxylated quaternary ammonium compounds is a compound according to formula (II):

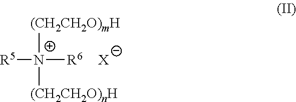

wherein R⁵ represents C₆-C₂₂ hydrocarbyl, preferably C₆-C₂₂ alkyl or alkenyl, more preferably C₈-C₂₀ alkyl or alkenyl, and most preferably C₁₀-C₁₈ alkyl or alkenyl;
R⁶ represents C₁-C₃ hydrocarbyl, preferably C₁-C₃ alkyl;
m and n are non-zero integers, the sum of m and n being in the range of from 2 to 20;
X⁻ is an anion, preferably a halide, such as chloride; an acetate or methyl acetate; or alkylsulphate, such as methyl- or ethylsulphate;
or according to formula (III)

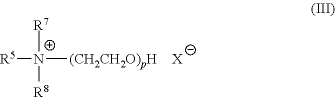

Wherein R⁵ represents C₆-C₂₂ hydrocarbyl, preferably C₆-C₂₂ alkyl or alkenyl, more preferably C₈-C₂₀ alkyl or alkenyl, and most preferably C₁₀-C₁₈ alkyl or alkenyl;
R⁷ and R⁸ represent independently C₁-C₃ hydrocarbyl, preferably C₁-C₃ alkyl;
p is an integer ranging from 2 to 20;
X⁻ is an anion, preferably a halide, such as chloride; an acetate or methyl acetate; or alkylsulphate, such as methyl- or ethylsulphate.

Nonionic surfactants that can be used in accordance with this invention as an ingredient of the fabric softener composition include alcohol ethoxylates and alkyl glucoside. Specifically, the nonionic surfactants suitable for use are commercially available as, for example, Berol® 175, AG® 6206, from Akzo Nobel.

Alkylaryl sulfonates that can be used in accordance with this invention as an ingredient of the fabric softener composition include, but not limited to, sodium xylene sulfonate and sodium cumene sulfonate. Specifically, one of the alkylaryl sulfonates suitable for use is commercially available as, for example, Witconate® SXS from Akzo Nobel.

Alkylsulfonate compounds that can be used in accordance with this invention as an ingredient of the fabric softener composition include, but not limited to, sodium C₁₄-C₁₆ olefin sulfonate, sodium octane sulfonate. Specifically, the alkylsulfonate compounds suitable for use are commercially available as, for example, Witconate® AOS, Witconate® NAS-8 from Akzo Nobel.

In addition to the above discussed ingredients the fabric softener composition of the present invention may comprise other components that are conventionally known in the art. These may include: fatty alcohols, electrolytes, anti-foaming agents, stabilizers, fragrances, and colors.

In another aspect of the present invention, use of the fabric softener composition of the present invention is provided. Specifically, the composition is capable of counteracting the harsh feel of fabrics such as cotton fabric, and therefore and be widely used in, but not limited to, fabric caring applications, hair conditioning applications, and anti-static applications.

### EXAMPLES

The invention will be elucidated with reference to the following examples. These are intended to illustrate the invention but are not to be construed as limiting in any manner the scope thereof.

The fabric softener compositions according to the present invention were prepared according to Table 1 below which includes different formulations for examples 1 to 3.

TABLE 1
Formulations of examples 1-3

| | % by weight | | |
|---|---|---|---|
| Ingredient | Example 1 | Example 2 | Example 3 |
| Di(hydrogenated tallow) | 30 | 30 | 30 |
| dimethyl ammonium | | | |
| methylsulfate | | | |
| isopropanol | 50 | 50 | 50 |
| coco | 1 | | |
| alkylbis(hydroxyethyl)methyl, | | | |
| ethoxylated quaternary | | | |
| ammonium compounds, | | | |
| chlorides, the number of EO | | | |
| units on average being 17 | | | |
| C₁₂₋₁₆ Alcohol ethoxylate, the | | 1 | |
| number of EO units on | | | |
| average being 8 | | | |
| C₆ Alkyl glucoside | | | 1 |
| Citric acid | pH to 4 | pH to 4 | pH to 4 |
| DI water | to 100 | to 100 | to 100 |

The demonstrated fabric softener compositions in the table above were observed to have a light yellow, clear and stable liquid appearance at a temperature not less than 25° C., and are able to pass 3 cycles of freeze-thaw (2-25° C.) stability test.

The compositions obtained according to examples 1-3 were used in combination with other ingredients to produce final product of fabric softeners (either regular or concentrate version) by cold process preparation without heating.

5-9 wt % based on the total weight of the final product of the compositions obtained according to examples 1-3 were respectively mixed with fragrance and colorant as desired, and balance deionized water, as specified in the following steps.

1) Adding approximately 50 wt % of the total weight of the final product of cold deionized water into a container;

2) Adding the desired amount of colorant/dye into the water, and agitating to obtain a mixture solution;

3) Dispersing one of the compositions obtained according to examples 1-3 into the water with moderate agitation until the lumps are absent;

4) Adding the balance of the cold deionized water;

5) Adding the desire amount of fragrance and optional optical brightener. slowly agitating to ensure proper dispersion of the fragrance and optical brightener;

6) Adding desire amount of electrolytes to lower the viscosity if necessary;

Key benefits for the example compositions:

The compositions of examples 1-3 were found to be suitable for use in the cold-water dispersing process, without heating operation required at all. In addition, all the steps were simply handled in liquid phase only.

1. A fabric softener composition which is in liquid state, comprising:
30-70 wt % based on the total weight of the composition of a quaternary ammonium compound according to formula (I): 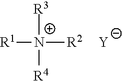
(I)
wherein R¹ and R² are independently a hydrocarbyl group having from 6 to 24, preferably from 8 to 22, more preferably from 12 to 20, most preferably from 14 to 18 carbon atoms;
R³ and R⁴ are independently selected from the group consisting of C₁to C₃ alkyl;
Y⁻ is an anion;
0.01-10 wt % based on the total weight of the composition of a compound selected from alkoxylated quaternary ammonium compounds,
0-70 wt % based on the total weight of the composition of water; and
1-60 wt % based on the total weight of the composition of an organic solvent selected from isopropanol, ethanol, benzyl alcohol, glycerin, propylene glycol and mixtures thereof.

2. The composition according to claim 1, wherein the composition comprises 0.01-5 wt % based on the total weight of the composition of the compound selected from alkoxylated quaternary ammonium compounds, nonionic surfactants, alkylaryl sulfonates, and alkylsulfonate compounds.

3. The composition according to claim 1, wherein the composition further comprises a pH adjusting agent so that the pH value of the composition is not greater than 7.

4. The composition according to claim 1, wherein the alkoxylated quaternary ammonium compounds have the following formula (II): 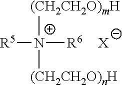
wherein R⁵ represents C₆-C₂₂ hydrocarbyl, preferably C₆-C₂₂ alkyl or alkenyl, more preferably C₈-C₂₀ alkyl or alkenyl, and most preferably C₁₀-C₁₅ alkyl or alkenyl;
R⁶ represents C₁-C₃ hydrocarbyl, preferably C₁-C₃ alkyl;
m and n are non-zero integers, the sum of m and n being in the range of from 2 to 20;
X⁻ represents an anion.

5. The composition according to claim 1, wherein the alkoxylated quaternary ammonium compounds have the following formula (III): 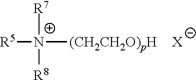
(III)
wherein R⁵ represents C₆-C₂₂ hydrocarbyl, preferably C₆-C₂₂ alkyl or alkenyl, more preferably C₈-C₂₀ alkyl or alkenyl, and most preferably C₁₀-C₁₈ alkyl or alkenyl;
R⁷ and R⁸ represent independently C₁-C₃ hydrocarbyl, preferably C₁-C₃ alkyl;
p is an integer ranging from 2 to 20;
X⁻ represents an anion.

6. A method for the treatment of a fabric, comprising the step of bringing said fabric in contact with the composition defined in claim 1.

7. A method for the treatment of hair, comprising the step of bringing said hair in contact with the composition defined in claim 1.

8. The use of a composition as defined in claim 1 as a fabric softener.

9. The use of a composition as defined in claim 1 as an anti-static agent.
